(19) United States
(12) Patent Application Publication
(10) Pub. No.: US 2020/0146557 A1
(43) Pub. Date: May 14, 2020

(54) Smart Body Temperature Screening System at Controlled Area

(71) Applicant: Electrical and Mechanical Services Department, The Government of the Hong Kong Special Administrativ, Hong Kong (HK)

(72) Inventors: Jovian Man Chit Cheung, Hong Kong (HK); Hiu Fai Siu, Hong Kong (HK); Richard Hau Yue So, Hong Kong (HK); Jing Wei Chin, Hong Kong (HK)

(21) Appl. No.: 16/679,349

(22) Filed: Nov. 11, 2019

Related U.S. Application Data
(60) Provisional application No. 62/766,886, filed on Nov. 9, 2018.

(51) Int. Cl. A61B 5/01; G06T 7/73; G06T 7/00; G06T 7/55; G06T 7/246; H04N 5/247; A61B 5/00

(52) U.S. Cl. CPC A61B 5/015; G06T 7/73; G06T 7/0012; G06T 7/55; G06T 7/246; H04N 5/247; A61B 2560/0252; G06T 2207/10024; G06T 2207/10048; G06T 2207/10016; G06T 2207/30201; G06T 2207/20081; A61B 5/7267

(57) ABSTRACT

The present invention provides a smart body temperature screening system (100) at controlled area and includes: images of controlled area (110); an IR image capturing and thermal analysis system (120) configured to take in digitized IR images sequences and determine the pixel coordinates with temperature higher than pre-determined thresholds; a color image capturing and location tracking system (130) configured to capture the color image sequence and determine the pixel coordinates of human, combine the pixel coordinates from the IR image capturing and thermal analysis system with the pixel coordinates of human to identify the pixel coordinates of feverish suspects with above threshold temperature; a facial image capturing system (140) configured to capture the pictures of the feverish suspects according to the pixel coordinates of feverish suspects and the original color images from the color image capturing and location tracking system; a virtual floor plan visualization system (150) configured to map the pixel coordinates of feverish suspects on to a virtual floor plan; a machine learning system (160) configured to use machine learning to minimize the influence of environmental factors and improve the accuracy for detecting the feverish persons from the feverish suspects. The present invention can smartly identify and locate fever suspects without locking the fever suspects in a specific area.

### CROSS REFERENCE TO RELATED APPLICATIONS

The present application claims the benefit of US provisional application No. 62/766,886 filed on Nov. 9, 2018, the contents of which are incorporated herein by reference in their entirety.

### FIELD OF THE INVENTION

The present invention generally relates to the technical field of designing image and/or video analysis systems, and in particularly, to a smart body temperature screening system at controlled area.

### BACKGROUND OF THE INVENTION

Body temperature is one of the indicators for the health status of a person. At control borders, thermal screening using infra-red (IR) cameras is a usual practice as a screening tool to identify those who have high fever. Typical setups consist of an IR video camera and a color video camera. The inspectors manually screen and determine the feverish suspects. This can cause visual stress and involve human errors.

In the prior art, the disclosure of CN108498080A discloses a smart health-care system for quick screening feverish suspects for public health management. The smart health-care system includes a remote system management platform, multiple regional screening systems, and multiple field disposal systems communicating with each regional screening system. The regional screening systems screen human body temperature, confirm abnormal human body objects with abnormal body temperature, detects abnormal human body object's physical signs and identifies key monitoring objects in abnormal human objects. The on-site disposal system makes control measures for key monitored items. The remote system management platform receives abnormal human objects, key monitoring objects, physical signs of detection and records of control measures, and makes tracking and statistical services. It has two main unique characteristics: (i) using IR thermal cameras to detect the body temperature of human inside a locked area (the detection zone); and (ii) release the lock when the person has no fever. However, human must be locked in a specific area in the prior art.

In another prior art, the disclosure of CN104594754A indicates a smart rapid automatic screening system for infectious diseases in public places. The smart rapid automatic screening system includes a main controller, which is connected with an image processing system. The image processing system is connected with infrared thermal image sensor, high definition digital camera and work indicator. The main controller is also connected with an alarm device. The main controller is also connected with the direction indicator. The display board is connected, and the main controller is also connected with at least one automatic door. When an outsider enters and approaches the invention, the infrared thermal image sensor starts to detect, and after obtaining the result, it responds through the main controller. If the temperature of the person's body surface is normal, it directly enters the public area. If the person's surface temperature is abnormal, it responds through the main controller, and notifies the CDC personnel. Meanwhile the camera records the image information of the person, prompts the direction of the person's walking, enters the isolation area, and the CDC personnel conduct a detailed inspection. It has two main unique characteristics: (i) using IR thermal cameras to detect the body temperature of human inside a locked area (the detection zone); and (ii) release three sets of locked doors when the person has no fever. However, it also needs to lock human in a specific area.

### SUMMARY OF THE INVENTION

In view of the above-mentioned problems, the object of the present invention is to design a smart body temperature screening system at controlled area capable of identifying and screening smartly feverish suspects with body temperature exceeding predetermined thresholds.

Another object is to capture color videos of crowd flow and capture thermal videos of crowd flow at the same time.

Another object is to identify suspects with high body temperature and track the suspect's locations and movements.

Another object is to visualize the suspect's locations on a virtual floor plan.

Another object is to record and monitor the accuracies associated with feverish suspect's identification.

Another object is to record and monitor the accuracies associated with feverish suspect's positions and movements.

Another object is to record the video data associated with the correct and false alarms in feverish suspect's identification.

One more object is to record the video data associated with the tracking of feverish suspects.

Another object is to use machine learning to minimize the influence of environmental factors and improve the accuracy for detecting the feverish persons from the feverish suspects.

For achieving the above objects, the embodiment of the present invention provides a smart body temperature screening system at controlled area, including: images of controlled area, an IR image capturing and thermal analysis system configured to take in digitized IR images sequences and determine the pixel coordinates with temperature higher than pre-determined thresholds; a color image capturing and location tracking system configured to capture the color image sequence and determine the pixel coordinates of human, combine the pixel coordinates from the IR image capturing and thermal analysis system with the pixel coordinates of human to identify the pixel coordinates of feverish suspects with above threshold temperature; a facial image capturing system configured to capture the pictures of the feverish suspects according to the pixel coordinates of feverish suspects and the original color images from the color image capturing and location tracking system; a virtual floor plan visualization system configured to map the pixel coordinates of feverish suspects on to a virtual floor plan; a machine learning system configured to use machine learning to minimize the influence of environmental factors and improve the accuracy for detecting the feverish persons from the feverish suspects.

For achieving the above objects, a smart body temperature screening method based on the smart body temperature screening system comprises: capturing color videos of crowd flow; capturing thermal videos of crowd flow; identifying feverish suspects with above threshold temperature; tracking locations and movements of feverish suspects; visualizing locations of the feverish suspects on the virtual floor plan; recording and monitoring the accuracies associated with identification of the feverish suspects; recording and monitoring the accuracies associated with positions and movements of the feverish persons; recording the video data associated with correct and false alarms in identification of the feverish suspects; recording the video data associated with tracking of feverish suspects; and using machine learning to minimize the influence of environmental factors and improve the accuracy for detecting the feverish persons from the feverish suspects.

Moreover, the identification is an ID number assigned to each feverish suspect identified by the system, wherein the identification information contains an ID, photos of the feverish suspects, and their coordinates on the virtual floor plan. The users find the feverish suspects according to the identification information and then manual check their temperature again; if the results of manual checking match with the system's detection decision rules, the feverish suspects are identified as feverish persons; if the results of manual checking match with the system's detection decision rules, the feverish suspects are identified as normal and they are the near-miss cases. the system records and monitors positions and movements of the feverish suspects until the feverish suspects moved out of the controlled area; when the feverish suspects move out of the controlled area, the locations of feverish suspects are broadcasted through mobile devices and tracked by the users, so that the feverish suspects do not need to be locked in a specific area.

The present invention can determine the pixel coordinates of the feverish suspects whose temperature is higher than the predetermined thresholds through the IR image capturing and thermal analysis system, determine the pixel coordinates of the human image through the color image capturing and location tracking system, and combine the pixel coordinates of the feverish suspects whose temperature is higher than the predetermined thresholds with the pixel coordinates of the human image, so as to automatically recognize the pixel coordinates of the human image locate the feverish suspects and in the controlled area. In addition, the location of feverish suspects can be broadcasted through mobile devices and tracked by operators, so that the locations of feverish suspects can be tracked outside the controlled area and the feverish suspects no need to be locked in a specific area.

### BRIEF DESCRIPTION OF THE DRAWINGS

To better understand the nature and advantages of the present invention, reference should be made to the following description and the accompanying figures. It should be understood, however, that each of the figures is provided for the purpose of illustration only and is not intended as a definition of the limits of the scope of the present invention. Also, as a general rule, and unless it is evident to the contrary from the description, where elements in different figures use identical reference numbers, the elements are generally either identical or at least similar in function or purpose.

FIG. 1 is a schematic view of smart body temperature screening system at controlled area according to the present invention;

FIG. 2 is a schematic view of smart body temperature screening method at controlled area according to the present invention.

### DETAILED DESCRIPTION OF THE INVENTION

The present invention would be further described herein with reference to the accompanying drawings and embodiments of the present invention. While example embodiments may include various modifications and alternative forms, embodiments thereof are shown by way of example in the drawings and will herein be described in detail. It should be understood, however, that there is no intent to limit example embodiments to the particular forms disclosed, but on the contrary, example embodiments are to cover all modifications, equivalents, and alternatives falling within the scope of the claims. Like numbers refer to like elements throughout the description of the figures.

It is to be understood that terms such as “left,” “right,” “top,” “bottom,” “front,” “rear,” “side,” “height,” “length,” “width,” “upper,” “lower,” “interior,” “exterior,” “inner,” “outer” and the like as may be used herein, merely describe points or portions of reference and do not limit the present invention to any particular orientation or configuration. Further, terms such as “first,” “second,” “third,” etc., merely identify one of a number of portions, components and/or points of reference as disclosed herein, and do not limit the present invention to any particular configuration or orientation.

Preferred embodiment of present invention provides a smart body temperature screening system at controlled area. The flow and structure of the embodiment for IR image capturing and thermal analysis module, color image capturing and suspect's location analysis module, suspect's facial image capturing module, virtual floor plan broadcasting and visualization module and machine learning system are described below in connection with the drawings.

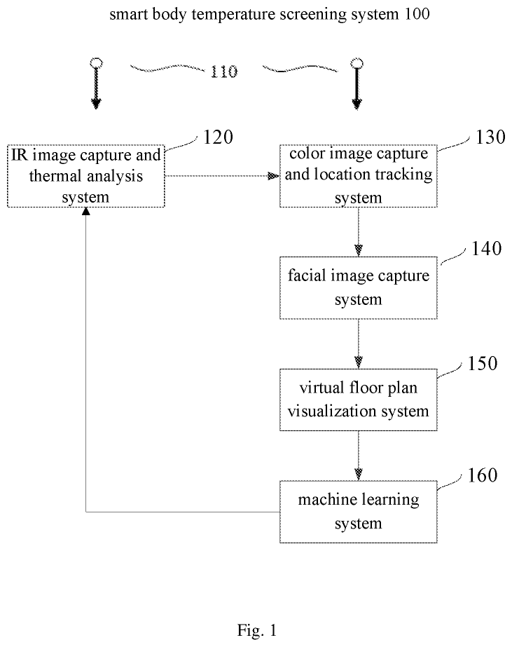

FIG. 1 is a schematic view of smart body temperature screening system at controlled area according to the present invention. The system includes: images of controlled area 110, an IR image capturing and thermal analysis system 120, a color image capturing and location tracking system 130, a facial image capturing system 140, a virtual floor plan visualization system 150, and machine learning system to minimize the influence of environmental factors 160.

The images of controlled area 110 include thermal IR images captured by the IR image capturing and thermal analysis system 120 and color images captured by the color image capturing and location tracking system 130. Both images are images of the same controlled area 110. The differences are the IR images are sensitive to IR light, while the color images are sensitive to visible light.

The IR image capturing and thermal analysis system 120 is configured to take in digitized IR images sequences and determine the pixel coordinates with temperature higher than pre-determined thresholds, and transmit the pixel coordinates to the color image capturing and location tracking system 130.

The color image capturing and location tracking system 130 is configured to capture the color image sequence and determine the pixel coordinates of human, combine the pixel coordinates from the IR image capturing and thermal analysis system 120 with the pixel coordinates of human to identify the pixel coordinates of feverish suspects with above threshold temperature, and transmit the pixel coordinates of feverish suspects and the original color images to the facial image capturing system 140.

The facial image capturing system 140 is configured to capture the pictures of the feverish suspects according to the pixel coordinates of feverish suspects and the original color images from the color image capturing and location tracking system 130, and transmit the pixel coordinates of feverish suspects and the pictures of the feverish suspects to the virtual floor plan visualization system 150. Specifically, the pixel coordinates of the estimated facial area of the feverish suspect is mapped to the color image pixel to copy out as the pictures of the feverish suspects.

The virtual floor plan visualization system 150 is configured to map the pixel coordinates of feverish suspects on to a virtual floor plan. Specifically, the depth of the feverish suspect is estimated by a combination of binocular disparity information. Pixel coordinates of the feet and face of the feverish suspect are captured by the color image capturing and location tracking system 130. Once the depth and the pixel coordinates are known, the pixel coordinates of the feverish suspect can be readily mapped on to a unique point on the virtual floor plan. The assumptions here are: all cameras in the IR image capturing and thermal analysis system 120 and the color image capturing and location tracking system 130 do not move and the floor plan is flat and level.

The machine learning system 160 is configured to analyze the training data by the machining learning algorithms and feedback the specific settings to the IR image capturing and thermal analysis system 120 to minimize the influence of environmental factors and improve the accuracy for detecting the feverish persons. Specifically, the machine learning system 160 analyzes all the near-miss cases. The near-miss cases refer to false negative, i.e. cases in which someone with fever but passed through manual checking. Machining learning algorithms required training data. In the embodiment of the present invention, the training data are the recorded cases in which the persons are detected as the feverish suspect but are rejected by the system's detection decision rules. Since the thermal IR images captured by the IR image capturing and thermal analysis system 120 and the color images captured by the color image capturing and location tracking system 130 are all recorded. The machine learning system analyzes and learns those training data from the recorded images that are nearly identified as feverish but finally decided to normal to see whether the decision rules should be further optimized. This is repeated daily to reduce the number of near-missed cases.

Another embodiment of present invention provides a smart body temperature screening method based on the above-mentioned smart body temperature screening system.

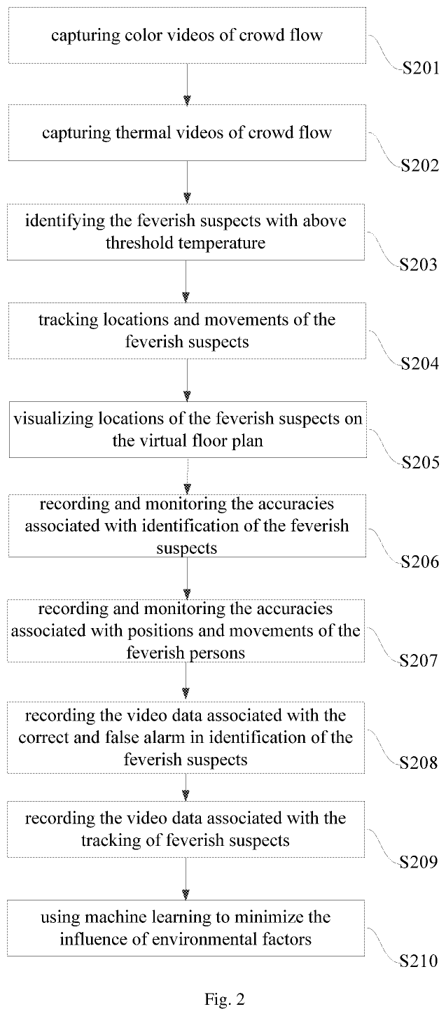

FIG. 2 is a schematic view of smart body temperature screening method according to the present invention. The method includes:

S201, capturing color videos of crowd flow.

In the step, the system captures the color image sequences of crowd flow and determines the pixel coordinates of human.

S202, capturing thermal videos of crowd flow.

In the step, the system captures the IR images sequences and determines the pixel coordinates with temperature higher than predetermined thresholds. In the embodiment of the present invention, the gray scales of the images are only sensitive to thermal radiation with light means hotter and dark means colder.

S203, identifying the feverish suspects with above threshold temperature.

In the step, the system identifies the pictures of the feverish suspects according to the pixel coordinates of feverish suspects and the original color images.

S204, tracking locations and movements of the feverish suspects.

In the step, movements are defined as time stamped position over a period of time. The system tracks the feverish suspect's locations and movements according to the pixel coordinates of feverish suspects.

S205, visualizing locations of the feverish suspects on the virtual floor plan.

In the step, the system maps the pixel coordinates of feverish suspects on to a virtual floor plan to visualize the feverish suspect's location and movements.

S206, recording and monitoring the accuracies associated with identification of the feverish suspects.

In the step, the identification is an ID number assigned to each feverish suspect identified by the system. The identification information contains an ID, photos of the suspect's faces, and their coordinates on the virtual floor plan. The users find the feverish suspects according to the identification information and then manual check their temperature again. If the results of manual checking match with the system information, then it is accurate. If not, it is inaccurate, i.e. the near-miss cases.

S207, recording and monitoring the accuracies associated with positions and movements of the feverish persons.

In the step, after identifying the feverish persons from the feverish suspects, the system records and monitors positions and movements of the feverish persons.

S208, recording the video data associated with the correct and false alarm in identification of the feverish suspects.

In the step, the system identifies the feverish suspects with above threshold temperature and monitors their positions and movements. The users find the feverish suspects according to the identification information and then manual check their temperature again to confirm the feverish persons. True positive result, i.e. the feverish persons from the feverish suspects after manual checking, means the correct alarms. False negative, i.e. unfeverish persons from the feverish suspects after manual checking, means the false alarms. The system records the video data associated with the correct and false alarms in feverish suspect's identification. Alternatively, all videos are recorded but are subject to deletion except they are related to the correct and false alarms.

S209, recording the video data associated with the tracking of feverish suspects.

In the step, the video data associated with the tracking of feverish suspects are kept as the historical records once the feverish suspects are identified by the system until the feverish suspects moved out of the controlled area.

S210, using machine learning to minimize the influence of environmental factors.

In the step, the recorded videos data associated with feverish suspects can be used as training data for machine learning. The system analyzes the training data by the machining learning algorithms and feedback the specific settings to minimize the influence of environmental factors and improve the accuracy for detecting the feverish persons from the feverish suspects.

The smart body temperature screening system of the present invention can be used in the controlled area, and also can track the positions of feverish suspects outside the controlled area. Specifically, the location of feverish suspects is broadcast through mobile devices and tracked by operators. The present invention is different from those prior art in that the system of the present invention does not need require to lock human in a confined area. The system of the present invention can automatically identify and locate the feverish suspects without locking the feverish suspect in a specific area.

It is to be understood that the embodiment of the present invention which has been described is merely illustrative of one application of the principles of the invention. Numerous modifications may be made to the specific structures and functions used in that embodiment without departing from the true spirit and scope of the invention.

What is claimed is:

1. A smart body temperature screening system at controlled area, characterized in that comprises:
images of controlled area;
an IR image capturing and thermal analysis system configured to take in digitized IR images sequences and determine the pixel coordinates with temperature higher than pre-determined thresholds;
a color image capturing and location tracking system configured to capture the color image sequence and determine the pixel coordinates of human, combine the pixel coordinates from the IR image capturing and thermal analysis system with the pixel coordinates of human to identify the pixel coordinates of feverish suspects with above threshold temperature;
a facial image capturing system configured to capture the pictures of the feverish suspects according to the pixel coordinates of feverish suspects and the original color images from the color image capturing and location tracking system;
a virtual floor plan visualization system configured to map the pixel coordinates of feverish suspects on to a virtual floor plan;
a machine learning system configured to use machine learning to minimize the influence of environmental factors and improve the accuracy for detecting the feverish persons from the feverish suspects.

2. The smart body temperature screening system according to claim 1, wherein the images of controlled area include thermal IR images captured by the IR image capturing and thermal analysis system and color images captured by the color image capturing and location tracking system, the IR images are sensitive to IR light, while the color images are sensitive to visible light.

3. The smart body temperature screening system according to claim 1, wherein the facial image capturing system receives the pixel coordinates of feverish suspects and the original color images from the color image capturing and location tracking system, the pixel coordinates of the estimated facial area of the feverish suspects are mapped to the color image pixel to copy out as the pictures of the feverish suspects.

4. The smart body temperature screening system according to claim 1, wherein the virtual floor plan visualization system estimates the depth of the feverish suspects according to binocular disparity information and combines pixel coordinates of the feet and face of the feverish suspects captured by the color image capturing and location tracking system, to map the pixel coordinates of feverish suspects on to the virtual floor plan.

5. The smart body temperature screening system according to claim 1, wherein the machine learning system analyze the training data by the machining learning algorithms and feedback the specific settings to the IR image capturing and thermal analysis system to minimize the influence of environmental factors and improve the accuracy for detecting the feverish persons form the feverish suspects.

6. The smart body temperature screening system according to claim 5, wherein the machine learning system analyzes and learns all the near-missed cases in which the persons are detected as the feverish suspects but are decided to normal to optimize the system's decision rules to reduce the number of near-missed cases.

7. The smart body temperature screening system according to claim 1, wherein a smart body temperature screening method based on the smart body temperature screening system comprises:
capturing color videos of crowd flow;
capturing thermal videos of crowd flow;
identifying feverish suspects with above threshold temperature;
tracking locations and movements of feverish suspects;
visualizing locations of the feverish suspects on the virtual floor plan;
recording and monitoring the accuracies associated with identification of the feverish suspects;
recording and monitoring the accuracies associated with positions and movements of the feverish persons;
recording the video data associated with correct and false alarms in identification of the feverish suspects;
recording the video data associated with tracking of feverish suspects; and
using machine learning to minimize the influence of environmental factors and improve the accuracy for detecting the feverish persons from the feverish suspects.

8. The smart body temperature screening system according to claim 7, wherein the identification is an ID number assigned to each feverish suspect identified by the system, the identification information contains an ID, photos of the feverish suspects, and their coordinates on the virtual floor plan.

9. The smart body temperature screening system according to claim 8, wherein the users find the feverish suspects according to the identification information and then manual check their temperature again;
if the results of manual checking match with the system's detection decision rules, the feverish suspects are identified as feverish persons; if the results of manual checking match with the system's detection decision rules, the feverish suspects are identified as normal and they are the near-miss cases.

10. The smart body temperature screening system according to claim 9, wherein the system records and monitors positions and movements of the feverish suspects until the feverish suspects moved out of the controlled area;
when the feverish suspects move out of the controlled area, the locations of feverish suspects are broadcasted through mobile devices and tracked by the users, so that the feverish suspects do not need to be locked in a specific area.
